(19) United States
(12) Patent Application Publication
(10) Pub. No.: US 2004/0116796 A1
(43) Pub. Date: Jun. 17, 2004

(54) Methods and apparatus for scoring a substance

(76) Inventor: Jianying Li, New Berlin, WI (US)

Correspondence Address: John S. Beulick, Armstrong Teasdale LLP, Suite 2600, One Metropolitan Sq., St. Louis, MO, 63102, US

(21) Appl. No.: 10/322,122

(22) Filed: Dec. 17, 2002

(51) Int. Cl. A61B 5/05; G06F 17/00

(52) U.S. Cl. 600/407; 382/128; 128/922

(57) ABSTRACT

A method for evaluating substance scoring, the scoring based on imaging system-generated images of an object having regions of interest due to possible presence of the substance includes receiving a plurality of image data, processing the image data using a post-processing non-linear image filter, and scoring the processed image data.

### BACKGROUND OF THE INVENTION

This invention relates to computed tomographic (CT) imaging, and more particularly to methods and apparatus for generating a CT image calcification score.

It is known to use imaging data to identify evidence of disease by detecting and quantifying, i.e. “scoring”, substances that may be present in a patient's system. One known software system, for example, analyzes CT images of the heart to quantify amounts of calcium in coronary regions of interest. Scoring is based upon the volume and Hounsfield unit of a calcified region. A number called the “calcium score” expresses the quantity of calcium present in the patient's arterial system.

Since calcification scoring is used primarily as a screening test, reducing a dosage of radiation administered to a patient during a CT scan is desirable. At least one known method of calcium scoring uses the Agatston score which is susceptible to image noise. Accordingly, to reduce the image noise for low dose CT scans, the variation of the Agatston scores should also be reduced. At least one known imaging system uses a medium filter to reduce the image noise for images obtained with lower dose scans. However, the imaging system also produces inaccurate Agatston scores, i.e. lower scores, compared with those obtained with higher dose CT scans.

### BRIEF DESCRIPTION OF THE INVENTION

In one aspect, a method for evaluating substance scoring is provided. The scoring based on imaging system-generated images of an object having regions of interest due to possible presence of the substance. The method includes receiving a plurality of image data, processing the image data using a post-processing non-linear image filter, and scoring the processed image data.

In another aspect, a computed tomographic (CT) imaging system for evaluating calcium scoring is provided. The scoring is based on imaging system-generated images of an object having regions of interest due to possible presence of the calcium. The CT system includes a detector array comprising a plurality of detector cells, an x-ray source positioned to emit x-rays toward the detector array, and a processor operationally coupled to the detector array. The processor is configured to receive a plurality of projection data, reconstruct the plurality of image data, process the reconstructed image data using a post-processing non-linear image filter, and score the image data obtained using the post-processing non-linear image filter.

In a further aspect, a computer readable medium encoded with a program is provided. The medium is configured to instruct a computer to receive a plurality of image data, process the image data using a post-processing non-linear image filter, and score the processed image data.

### BRIEF DESCRIPTION OF THE DRAWINGS

FIG. 1 is a pictorial view of a CT imaging system.

FIG. 2 is a block schematic diagram of the system illustrated in FIG. 1.

FIG. 3 illustrates a method for evaluating substance scoring.

FIG. 4 illustrates a gain factor curve.

FIG. 5 illustrates a scoring comparison between a known calcium scoring method and method illustrated in FIG. 3.

### DETAILED DESCRIPTION OF THE INVENTION

In some known CT imaging system configurations, an x-ray source projects a fan-shaped beam which is collimated to lie within an X-Y plane of a Cartesian coordinate system and generally referred to as an “imaging plane”. The x-ray beam passes through an object being imaged, such as a patient. The beam, after being attenuated by the object, impinges upon an array of radiation detectors. The intensity of the attenuated radiation beam received at the detector array is dependent upon the attenuation of an x-ray beam by the object. Each detector element of the array produces a separate electrical signal that is a measurement of the beam attenuation at the detector location. The attenuation measurements from all the detectors are acquired separately to produce a transmission profile.

In third generation CT systems, the x-ray source and the detector array are rotated with a gantry within the imaging plane and around the object to be imaged such that the angle at which the x-ray beam intersects the object constantly changes. A group of x-ray attenuation measurements, i.e., projection data, from the detector array at one gantry angle is referred to as a “view”. A “scan” of the object comprises a set of views made at different gantry angles, or view angles, during one revolution of the x-ray source and detector.

In an axial scan, the projection data is processed to construct an image that corresponds to a two dimensional slice taken through the object. One method for reconstructing an image from a set of projection data is referred to in the art as the filtered back projection technique. This process converts the attenuation measurements from a scan into integers called “CT numbers” or “Hounsfield units”, which are used to control the brightness of a corresponding pixel on a cathode ray tube display.

To reduce the total scan time, a “helical” scan may be performed. To perform a “helical” scan, the patient is moved while the data for the prescribed number of slices is acquired. Such a system generates a single helix from a one fan beam helical scan. The helix mapped out by the fan beam yields projection data from which images in each prescribed slice may be reconstructed.

Reconstruction algorithms for helical scanning typically use helical weighing algorithms that weight the collected data as a function of view angle and detector channel index. Specifically, prior to a filtered backprojection process, the data is weighted according to a helical weighing factor, which is a function of both the gantry angle and detector angle. The helical weighting algorithms also scale the data according to a scaling factor, which is a function of the distance between the x-ray source and the object. The weighted and scaled data is then processed to generate CT numbers and to construct an image that corresponds to a two dimensional slice taken through the object.

As used herein, an element or step recited in the singular and preceded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Also as used herein, the phrase “reconstructing an image” is not intended to exclude embodiments of the present invention in which data representing an image is generated but a viewable image is not. However, many embodiments generate (or are configured to generate) at least one viewable image.

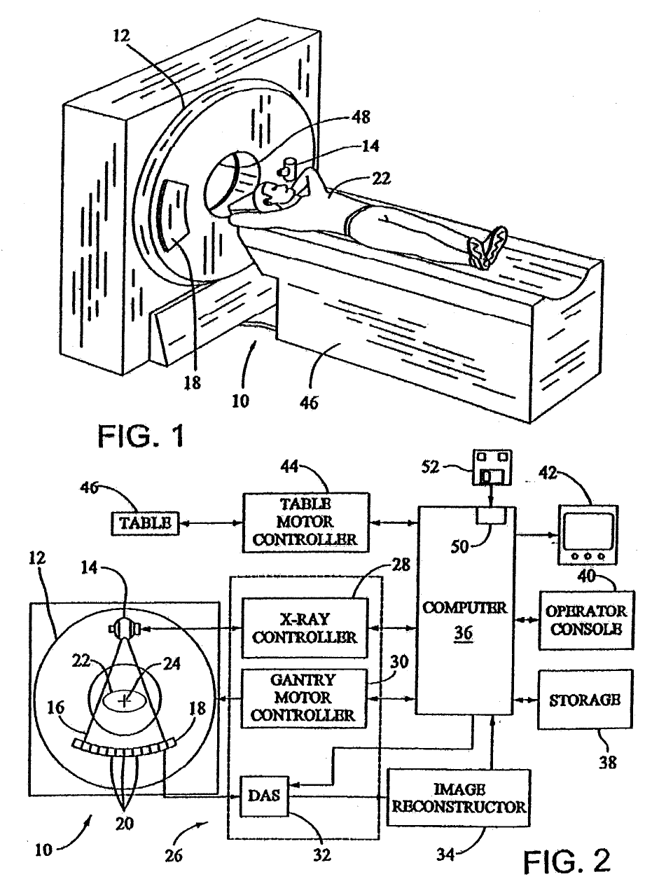

Referring to FIGS. 1 and 2, a multi-slice scanning imaging system, for example, a computed tomography (CT) imaging system 10, is shown as including a gantry 12 representative of a “third generation” CT imaging system. Gantry 12 has an x-ray source 14 that projects a beam of x-rays 16 toward a detector array 18 on the opposite side of gantry 12. Detector array 18 is formed by a plurality of detector rows (not shown) including a plurality of detector elements 20 which together sense the projected x-rays that pass through an object, such as a medical patient 22. Each detector element 20 produces an electrical signal that represents the intensity of an impinging x-ray beam and hence the attenuation of the beam as it passes through object or patient 22. During a scan to acquire x-ray projection data, gantry 12 and the components mounted thereon rotate about a center of rotation 24. FIG. 2 shows only a single row of detector elements 20 (i.e., a detector row). However, multislice detector array 18 includes a plurality of parallel detector rows of detector elements 20 such that projection data corresponding to a plurality of quasi-parallel or parallel slices can be acquired simultaneously during a scan.

Rotation of gantry 12 and the operation of x-ray source 14 are governed by a control mechanism 26 of CT system 10. Control mechanism 26 includes an x-ray controller 28 that provides power and timing signals to x-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high-speed image reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

In one embodiment, computer 36 includes a device 50, for example, a floppy disk drive, CD-ROM drive, DVD drive, magnetic optical disk (MOD) device, or any other digital device including a network connecting device such as an Ethernet device for reading instructions and/or data from a computer-readable medium 52, such as a floppy disk, a CD-ROM, a DVD or an other digital source such as a network or the Internet, as well as yet to be developed digital means. In another embodiment, computer 36 executes instructions stored in firmware (not shown). Computer 36 is programmed to perform functions described herein, and as used herein, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein.

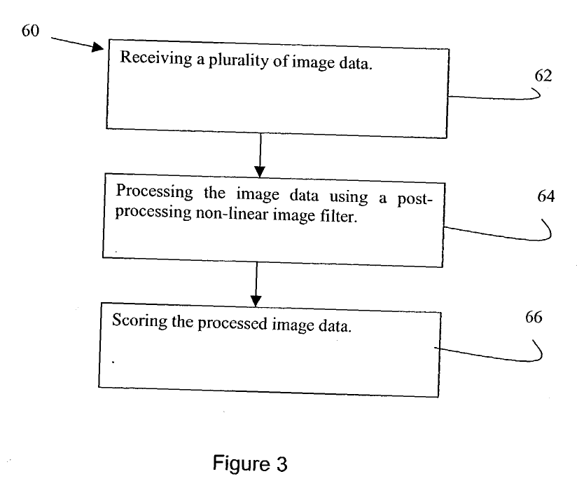

FIG. 3 illustrates a method 60 for evaluating substance scoring. In the exemplary embodiment, the substance is a calcification and the scoring is based on imaging system-generated images of an object 22 having regions of interest due to possible presence of the substance. Method 60 includes receiving 62 a plurality of image data, processing 64 the image data using a post-processing non-linear image filter, and scoring 66 the processed image data.

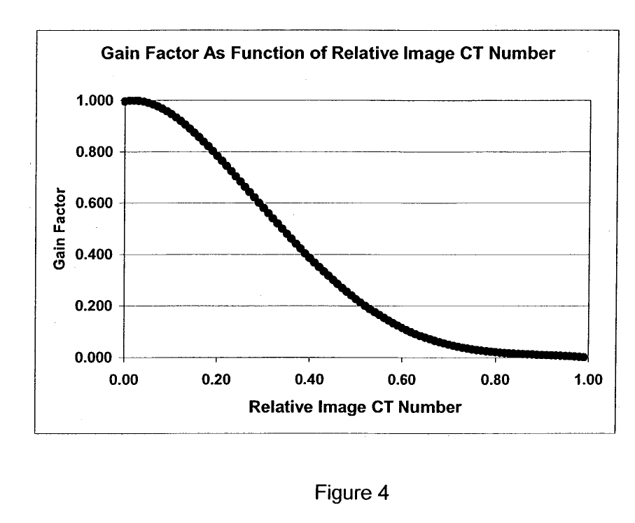

In use, a gain factor function is pre-generated and a threshold value T is pre-determined. The gain factor curve controls the degree of smoothing on the images and in general needs to be smooth and has high values (closer to 1) for low CT numbers and approaches to 0 for high CT numbers. Threshold value T determines the values of the gain factor curve. The gain factor function and the initial value of the threshold T are determined based on both high dose (low noise) and low dose (high noise) CT phantom scans to ensure that accurate scores are obtained for the low dose scans with the post-processing filter compared to those obtained with the high dose scans. The phantom contains known quantity of calcium inserts, so that quantitative assessment can be done in terms of the score accuracy. Threshold value T can then be adjusted later by the users if desired. In the exemplary embodiment, the gain factor curve is a function of the relative pixel value, Pᵣ(i, j) for image pixel P (i, j). FIG. 4 illustrates an exemplary embodiment of the gain factors as a function of the image CT number curve. An effective pixel value Pₑ(i, j) is then generated for each image pixel P(i, j) by averaging a plurality of pixels P(i, j) surrounding the image pixel P(i, j). For example, if four surrounding pixels are used, the effective pixel value Pₑ(i, j) can be calculated in accordance with:

Pₑ(i, j)=(P(i, j)+P(i−1, j)+P(i+1, j)+P(i, j−1)+P(i, j+1))/5 (1)

As described in Equation 1, four pixels surrounding each image pixel P(i, j), and the image pixel P(i, j) itself are added, and then divided by five to generate the effective pixel value Pₑ(i, j). Although the methods described herein use four surrounding pixels to generate each effective pixel value, a plurality of pixels other than four can be used to generate the effective pixel value Pₑ(i, j). For example, eight pixels, sixteen pixels, etc. are used in other embodiments. Also different weights may be used to change the contribution of the surrounding pixels to the final effective pixel value. Generating the effective pixel value Pₑ(i, j) facilitates reducing the impact of noisy image pixel values on the valuation of the gain factors. In an exemplary embodiment, an effective pixel value Pₑ(i, j) is generated for each image pixel P(i, j).

Using the determined threshold T and the effective pixel values, a relative pixel value can be calculated in accordance with:

Pᵣ(i, j)=Pₑ(i, j)/T (2)

The relative pixel value is then clipped to less or equal to 1.0 (i.e., if the relative pixel value for a particular pixel is greater than one, then the relative pixel value is set at one, otherwise if the calculated relative pixel value is less than one, the value is not adjusted.

Using the relative pixel values, a gain factor for each of the pixels is calculated using the gain factor curve shown in FIG. 4. For example:

Gain(i, j)=0.996+0.372*Pᵣ(i, j)−10.488*Pᵣ(i, j)²+19.044*Pᵣ(i, j)³−12.927*Pᵣ(i, j)⁴+3.003*Pᵣ(i, j)⁵ (3)

A final image pixel value P_{f}(i, j) for each image pixel P(i, j) is generated in accordance with:

P_{f}(i, j)=P(i, j)−(P(i, j)−smooth(P(i, j)))*Gain(i, j) (4)

where smooth(P(i, j)) is a conventional smoothing operation on the original pixel value P(i, j).

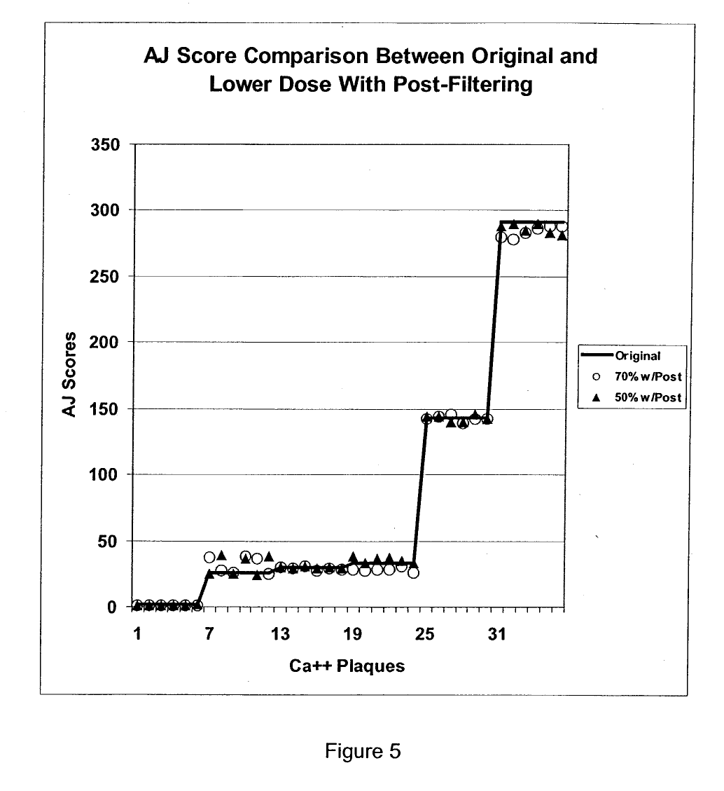

FIG. 5 illustrates a scoring comparison between a known calcium scoring method and method 60 described herein. As shown, calcium scoring accuracy is maintained while reducing a dosage of radiation to the patient.

In use, the post-processing non-linear image filter for calcification scoring describe herein, facilitates reducing image noise while maintaining a calcification score accuracy. Since, the smoothing is dependent on the CT numbers of the image pixel value, the pixels with lower CT numbers will get more smoothing, and the smoothness of individual pixels can be controlled simply by adjusting the threshold T.

While the invention has been described in terms of various specific embodiments, those skilled in the art will recognize that the invention can be practiced with modification within the spirit and scope of the claims.

What is claimed is:

1. A method for evaluating substance scoring, the scoring based on imaging system-generated images of an object having regions of interest due to possible presence of the substance, said method comprising:
receiving a plurality of image data;
processing the image data using a post-processing non-linear image filter; and
scoring the processed image data.

2. A method in accordance with claim 1 wherein said substance scoring comprises calcification scoring.

3. A method in accordance with claim 1 wherein scoring the image data processed using a post-processing non-linear image filter comprises:
determining a threshold value T;
generating an effective pixel value for each image pixel; and
calculating a relative pixel value for each image pixel using the determined threshold value and the effective pixel value.

4. A method in accordance with claim 3 further comprising calculating a gain factor for each relative pixel using a gain factor curve.

5. A method in accordance with claim 3 wherein calculating a relative pixel value comprises calculating a relative pixel value in accordance with:
Pᵣ(i, j)=Pₑ(i, j)/T;
where:
Pₑ(i, j) is an effective pixel value; and
T is a determined threshold value.

6. A method in accordance with claim 3 wherein generating an effective pixel value comprises generating an effective pixel value in accordance with:
Pₑ(i, j)=(P(i, j)+P(i−1, j)+P(i+1, j)+P(i, j−1)+P(i, j+1))/5
where:
P(i, j) is an image pixel P at position (i, j).

7. A method in accordance with claim 3 further comprising clipping the relative pixel value to less than or equal to one.

8. A method in accordance with claim 1 further comprising calculating a final image pixel value for each image pixel in accordance with:
P_{f}(i, j)=P(i, j)−(P(i, j)−smooth(P(i, j)))*Gain(i, j)
where:
smooth P(i, j) is a smoothing operation performed on each image pixel (P (i, j)); and
Gain(i, j) is a gain factor.

9. A method for evaluating calcification scoring, the scoring based on imaging system-generated images of an object having regions of interest due to possible presence of the calcification, said method comprising:
receiving a plurality of image data; and
scoring the image data processed using a post-processing non-linear image filter, wherein said scoring the image data includes:
determining a threshold value T;
generating an effective pixel value for each image pixel; and
calculating a relative pixel value for each image pixel using the determined threshold value and the effective pixel value.

10. A computed tomographic (CT) imaging system for evaluating calcium scoring, the scoring based on imaging system-generated images of an object having regions of interest due to possible presence of the calcium, said CT system comprising:
a detector array comprising a plurality of detector cells;
an x-ray source positioned to emit x-rays toward said detector array; and
a processor operationally coupled to said detector array, said processor configured to:
receive a plurality of projection data;
reconstruct the plurality of image data;
process the reconstructed image data using a post-processing non-linear image filter; and
score the image data obtained using the post-processing non-linear image filter.

11. A CT imaging system in accordance with claim 10 wherein said processor is further configured to:
receive a pre-determine a threshold value T;
generate an effective pixel value for each image pixel; and
calculate a relative pixel value for each image pixel using the pre-determined threshold value and the effective pixel value.

12. A CT imaging system in accordance with claim 10 wherein said processor is further configured to calculate a gain factor for each relative pixel using a gain factor curve.

13. A CT imaging system in accordance with claim 10 wherein to calculate a relative pixel value, said processor further configured to calculate a relative pixel value in accordance with:
Pᵣ(i, j)=Pₑ(i, j)/T;
where:
Pₑ(i, j) is an effective pixel value; and
T is a pre-determined threshold value.

14. A CT imaging system in accordance with claim 10 wherein said processor is further configured to clip the relative pixel value to less than or equal to one.

15. A CT imaging system in accordance with claim 10 wherein said processor is further configured to calculate a final image pixel value for each image pixel in accordance with:
P_{f}(i, j)=P(i, j)−(P(i, j)−smooth(P(i, j)))*Gain(i, j)
where:
smooth P(i, j) is a smoothing operation performed on each image pixel (P (i, j)); and
Gain(i, j) is a gain factor.

16. A CT imaging system in accordance with claim 15 wherein to generate an effective pixel value said processor further configured to generate an effective pixel value in accordance with:
Pₑ(i, j)=(P(i, j)+P(i−1, j)+P(i+1, j)+P(i, j−1)+P(i, j+1))/5
where:
P(i, j) is an image pixel P at position (i, j).

17. A CT imaging system in accordance with claim 13 wherein to generate an effective pixel value said processor further configured to generate an effective pixel value in accordance with:
Pₑ(i, j)=(P(i, j)+P(i−1, j)+P(i+1, j)+P(i, j−1)+P(i, j+1))/5
where:
P(i, j) is an image pixel P at position (i, j).

18. A computer readable medium encoded with a program configured to instruct a computer to:
receive a plurality of image data;
process the image data using a post-processing non-linear image filter; and
score the processed image data.

19. A computer readable medium in accordance with claim 18 further encoded to instruct the computer to:
receive a pre-determined threshold value T from a user;
generate an effective pixel value for each image pixel; and
calculate a relative pixel value for each image pixel using the determined threshold value and the effective pixel value.

20. A computer readable medium in accordance with claim 18 further encoded to instruct the computer to calculate a gain factor for each relative pixel using a gain factor curve.

21. A computer readable medium in accordance with claim 18 further encoded to instruct the computer to calculate a relative pixel value in accordance with:
Pᵣ(i, j)=Pₑ(i, j)/T;
where:
Pₑ(i, j) is an effective pixel value; and
T is a pre-determined threshold value.

22. A computer readable medium in accordance with claim 18 further encoded to instruct the computer to clip the relative pixel value to less than or equal to one.

23. A computer readable medium in accordance with claim 18 further encoded to instruct the computer to calculate a final image pixel value for each image pixel in accordance with:
P_{f}(i, j)=P(i, j)−(P(i, j)−smooth(P(i, j)))*Gain(i, j)
where:
smooth P(i, j) is a smoothing operation performed on each image pixel (P (i, j)); and
Gain(i, j) is a gain factor.

24. A computer readable medium in accordance with claim 23 further encoded to instruct the computer to generate an effective pixel value in accordance with:
Pₑ(i, j)=(P(i, j)+P(i−1, j)+P(i+1, j)+P(i, j−1)+P(i, j+1))/5
where:
P(i, j) is an image pixel P at position (i, j).

25. A computer readable medium in accordance with claim 18 further encoded to instruct the computer to generate an effective pixel value in accordance with:
Pₑ(i, j)=(P(i, j)+P(i−1, j)+P(i+1, j)+P(i, j−1)+P(i, j+1))/5
where:
P(i, j) is an image pixel P at position (i, j).
